(19) United States
(12) Patent Application Publication
(10) Pub. No.: US 2014/0257222 A1
(43) Pub. Date: Sep. 11, 2014

(54) Wound Cleaning Adhesive Patch

(71) Applicant: Peter R. Popa, Fort Lee, NJ (US)

(72) Inventor: Peter R. Popa, Fort Lee, NJ (US)

(21) Appl. No.: 14/280,583

(22) Filed: May 17, 2014

Related U.S. Application Data
(63) Continuation-in-part of application No. 13/198,576, filed on Aug. 4, 2011, now abandoned.

(51) Int. Cl. A61F 13/00; A61F 13/02

(52) U.S. Cl. CPC A61F 13/00063; A61F 13/0206; A61F 2013/0017
USPC 604/369; 604/367

(57) ABSTRACT

A patch that is able to expedite the healing process of skin wounds such as cuts, burns, scraps, or any other superficial wounds. The present invention is an adhesive patch with gauze retaining a suds substance. The patch is applied to a user's skin wound to clean and draw out the pus and exudates for a quicker healing process.

The current application is a continuation-in-part of and claims a priority to Ser. No. 13/198,576 filed on Aug. 4, 2011 which claims a priority to 61/371,476 filed on Aug. 6, 2010.

### FIELD OF THE INVENTION

The present invention relates generally to a wound patch. More specifically, the present invention is a skin wound patch for accelerated healing.

### BACKGROUND OF THE INVENTION

During the healing process of a regular skin wound, burn, open cut, etc. exudate, environmental contaminates, pus, etc., accumulate over the wound as a response to the injury. In most cases, people immediately apply antibiotics, dry sponge coverings, creams, oil based substances, etc. to the wound without removing any of the wound's waste material. Even after cleaning the wound with soap, water, and other substances, micro-contamination of the wound persists and continuously transudate, exudates, and possibly pus keep forming during the healing process. Because pus and/or exudates including bacteria, cellular debris, proteic components of inflammatory response, environmental particles, etc. slow the healing of the wound, draining of such components would remove a lot of potential harmful material sitting in the affected area. Therefore, removal of such is necessary in order to expedite the healing. The present invention uses gauze or sponge-like material soaked in soap-like or detergent-like suds to speed the healing process by removing the pus and waste. The surrounding adhesive film will stick on the healthy skin and will isolate the wound from the environment thereby preventing the suds from drying out and keeping the sponge or gauze in contact with the wound surface.

### BRIEF DESCRIPTION OF THE DRAWINGS

FIG. 1 is a perspective view of the present invention in a circular embodiment.

FIG. 2 is a perspective view of the present invention in a square embodiment.

FIG. 3 is a view of the present invention applied to a skin wound, wherein: the triangular particles are the exudates and pus from the skin wound; the circular particles are the suds substances; and the suds substances are shown dissolving and drawing the exudates away from the skin wound.

### DETAILED DESCRIPTIONS OF THE INVENTION

All illustrations of the drawings are for the purpose of describing selected versions of the present invention and are not intended to limit the scope of the present invention.

The present invention is a wound patch that is mostly intended but not restricted for superficial skin wounds such as abrasions, burns, etc. The present invention is especially useful for skin wounds with or without signs of infection. However, the wound patch is also used for the prevention of infection and wound cleaning where the skin barrier was broken and an inflammatory repairing process takes place.

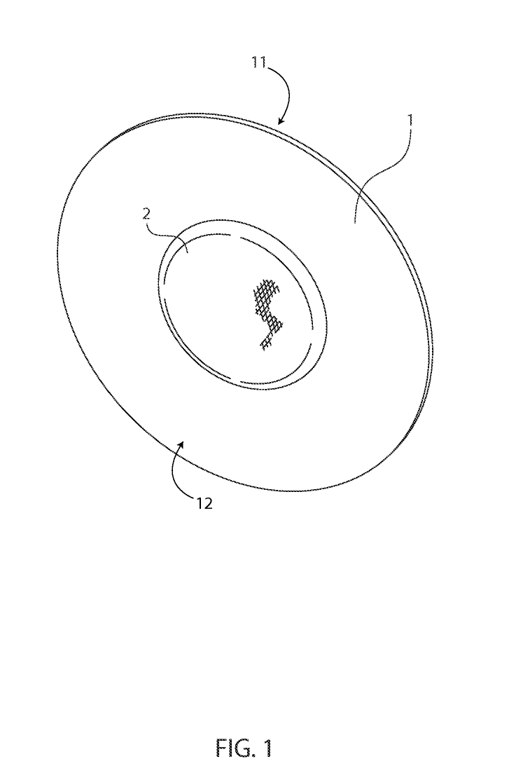

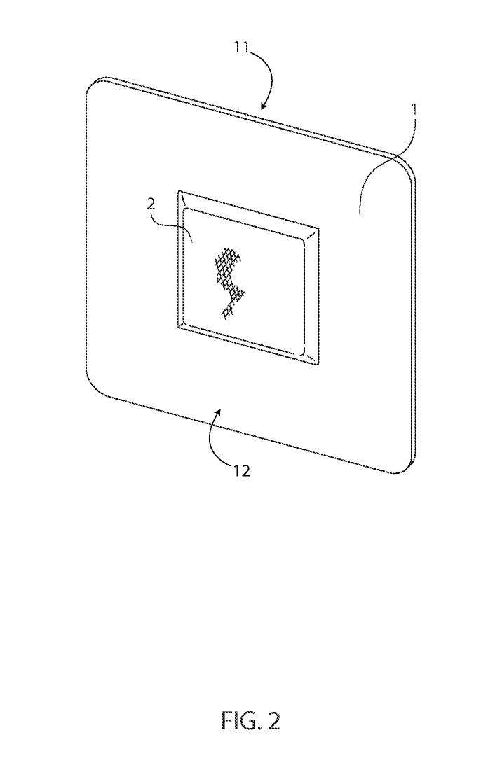

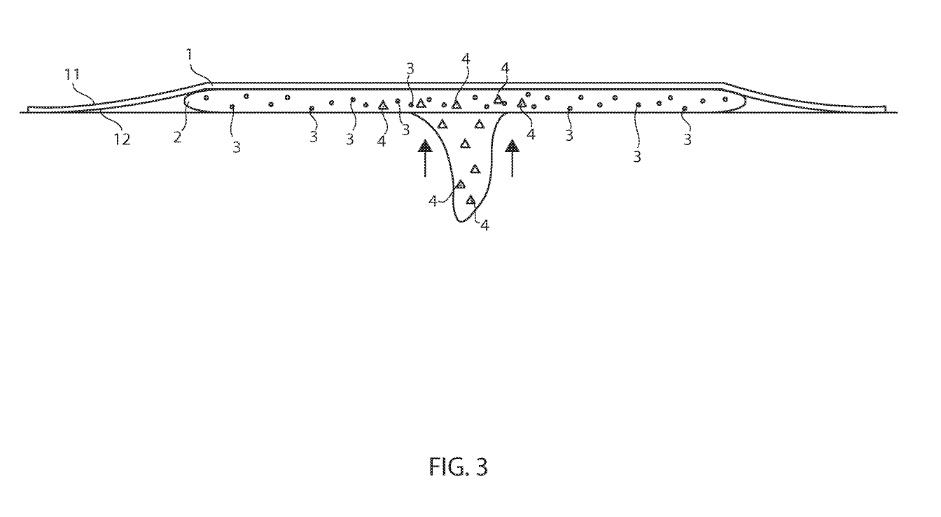

In reference to FIG. 1-3, the present invention is a wound cleaning adhesive patch that comprises an impermeable film 1, an absorbent pad 2, and a suds substance 3. The impermeable film 1 is used for the protection and sealing of the user's skin wound and comprises an adhesive surface 11 and a non-adhesive surface 12. The non-adhesive surface 12 is positioned above the adhesive surface 11. The absorbent pad 2 is adhered to and positioned on the adhesive surface 11 of the impermeable film 12. The absorbent pad 2 can be any absorbing material including gauze, dressing, sponge-like materials, or any other suitable absorbing material for application to wounds. The absorbent pad 2 is centrally adhered on the impermeable film 1 to ensure that the wound is completely sealed by the impermeable film 1. The absorbent pad 2 retains the suds substance 3, which can be any soap or detergent like solution which dissolves the above mentioned waste material and contaminates, solution that may or may not be antibacterial or contain antibiotic substances. The size and shape of the present invention can vary to accommodate the size and shapes of different wounds.

To use the present invention on a skin wound, it is important to perform a cleansing of the wound to remove any obvious external contaminants and dead tissue from the wound. The absorbent pad 2 is then aligned and placed over the wound. The adhesive surface 11 is then pressed against the user's skin in order to position the impermeable film 1 around the skin wound. The user can hold the impermeable film 1 with the non-adhesive surface 12 so that the adhesive surface 11 does not stick to their fingers. As a result, the wound and the absorbent pad 2 are sealed by the impermeable film 1 and prevent any additional bacteria, foreign substances, or other environmental factors from infecting and contaminating the wound. In addition, the adhesive surface 11 is a watertight sealant, which prevents those harmful factors from traveling through the contact surface between the impermeable film 1 and the user's skin. The sealing of the skin wound and the absorbent pad 2 by the impermeable film 1 also prevents the drying of the suds substance 3. The pus, exudates 4, etc., and other foreign substances, are drawn in by the suds substance 3 and the absorbent pad 2 by dissolving and diffusing it from a higher to lower concentration. As the wound excretes pus and/or exudates, the absorbent pad 2 is able to physically dissolve lipophilic material and remove it by diffusion, absorbing any dirt, cellular debris, proteic components from inflammatory responses, environmental particles, etc. from the surface of the wound. The suds substance 3 acts as a solvent facilitating the absorbing process through diffusion of the dissolved material from a higher to a lower concentration towards the pad. As a result, the pus and exudates 4 are drawn away from the skin wound, leaving the wound clean for an expedited healing process.

The principle of the present invention is the application of the suds substance 3 onto a wound by means of the absorbent pad 2. The suds substance 3 can be applied on the wound for a period of time under an occlusive dressing. This process will moisten the wound's fibrin crust and remove the pus, exudates 4, and other foreign substances from the wound by dissolving them into the suds substance 3 that are in contact with the surface of the wound. Even in the absence of any antibacterial properties of the applied soap/detergent suds, the application of the soap/detergent like material will have a beneficial healing effect by cleaning effectively the wound and helping the healing process.

The suds substance 3 should have the least amount of irritant properties to the skin and tissue so that the suds substance 3 can soothingly remove unwanted particles from the wound. The suds substance 3 removes these unwanted particles by forming micelles, which are spherical particles containing a lipophilic part and a hydrophilic part.

The lipophilic part bounds to the unwanted particles, which usually are insoluble fat/oil molecules, and the hydrophilic part shields the unwanted particles from the water so that the unwanted particles become soluble in water. Thus, the micelles will contain unwanted particles such as cellular debris, bacteria, etc., which will diffuse from a higher concentration (wound surface) to a lower concentration (absorbent pad 2). Consequently, the suds substance 3 should be a surfactant that is dissolved in sterile water. In the preferred embodiment of the present invention, the surfactant is mildly acidic and non-ionic in order to reduce the irritability to the skin and tissue, to protect the function of the underlying cells, and to protect the acid mantle for the healthy skin surrounding the wound. A pH of 5.5 is ideal for the suds substance 3, and the acidity of the surfactant should not decrease the pH of the suds substance 3 to less than 5.5. In addition, this kind of surfactant would not suppress the local immune response to the wound. Any surfactant can be chosen as long as the surfactant exhibits mildly acidic and non-ionic properties, some which include soap or a detergent.

The present invention has been tested on several patients with exceptional results. In one embodiment of the present invention, sterile gauze is scrubbed with a regular hypoallergenic soap to the desired scrub consistency. The patch is then applied to the wound and covered with the impermeable film 1. The impermeable film 1 can be a polymer film that is flexible and can easily conform to the user's skin. The impermeable film 1 is waterproof and moisture vapor impermeable to prevent outside contamination and drying of the absorbent pad 2. After 24 hours, upon the removal of the wound cleaning adhesive patch, the wound is left clean off all of the pus and exudates 4 which are dissolved into the suds substance 3 in the absorbent pad 2. The process can be repeated several times until the wound is considered clean. The present invention is extremely valuable also in providing symptom relief by decreasing pain and signs of inflammation due to removal of inflammatory proteins and thereof diminishing the nociceptive effect to provide symptom relief at the wound site.

The difference between the present invention and traditional wound dressings on the market is that the present invention applies a dressing containing soap/detergents like suds. The suds substances 3 contained in the absorbent pad 2 are able to drain and cleanse the wound. In comparison, wound dressings including ones that use antibiotics, dry sponge coverings, creams, alcohol or oil based substances do not facilitate pus, exudates, etc. 4 away from a skin wound.

In many cases, the infected open wounds require prescription of systemic antibiotics. The use of the present invention decreases the need for the use of systemic therapy. Additionally, with reduced usage of systemic antibiotics, the chances of bacterial resistance of developing are also decreased. Reduced antibiotic usage also decreases the money a patient must spend on antibiotics. However, in other cases, the present invention can also be used in conjunction with systemic antibiotic treatment by ameliorating symptoms, facilitating healing, and decreasing healing time. The present invention is also most efficient with use on wound with a larger superficial area, the present invention is still applicable to superficial small wounds or puncture wounds.

In another application of the present invention, the wound cleaning adhesive patch can be used for injuries such as burn wounds where the skin barrier has been compromised. Even if signs of infection are not visible, the removal of the exudates will deplete the bacteria from an excellent growth medium and in conjunction with the wound sealing will significantly decrease the chances of infection. Burn wounds are also superficial and can be prone to infection due to large amount of exudates/transudates 4.

Although the invention has been explained in relation to its preferred embodiment, it is to be understood that many other possible modifications and variations can be made without departing from the spirit and scope of the invention as hereinafter claimed.

What is claimed is:

1. A wound cleaning adhesive patch comprises:
an impermeable film;
an absorbent pad;
a suds substance;
the impermeable film comprises a non-adhesive surface and an adhesive surface;
the non-adhesive surface being positioned above the adhesive surface;
the absorbent pad being adhered to the impermeable film;
the absorbent pad being centrally positioned on the adhesive surface; and
the suds substance being retained by the absorbent pad.

2. The wound cleaning adhesive patch as claimed in claim 1, wherein the absorbent pad is selected from the group consisting of gauze, dressing, and sponges.

3. The wound cleaning adhesive patch as claimed in claim 1, wherein the impermeable film is a polymer film.

4. The wound cleaning adhesive patch as claimed in claim 1, wherein the adhesive surface is a watertight sealant.

5. The wound cleaning adhesive patch as claimed in claim 1, wherein the suds substance is at a pH of 5.5.

6. The wound cleaning adhesive patch as claimed in claim 1, wherein the suds substance is a surfactant dissolved in sterile water.

7. The wound cleaning adhesive patch as claimed in claim 6, wherein the surfactant is non-ionic.

8. The wound cleaning adhesive patch as claimed in claim 6, wherein the surfactant is slightly acidic.

9. The wound cleaning adhesive patch as claimed in claim 6, wherein the surfactant is selected from the group consisting of soap and detergent.

10. A wound cleaning adhesive patch comprises:
an impermeable film;
an absorbent pad;
a suds substance;
the impermeable film comprises a non-adhesive surface and an adhesive surface;
the non-adhesive surface being positioned above the adhesive surface;
the adhesive surface being a watertight sealant;
the absorbent pad being adhered to the impermeable film;
the absorbent pad being centrally positioned on the adhesive surface;
the suds substance being retained by the absorbent pad;
the suds substance being at a pH of 5.5;
the suds substance being a surfactant dissolved in sterile water; and
the surfactant being mildly acidic and non-ionic.

11. The wound cleaning adhesive patch as claimed in claim 10, wherein the absorbent pad is selected from the group consisting of gauze, dressing, and sponges.

12. The wound cleaning adhesive patch as claimed in claim 10, wherein the impermeable film is a polymer film.

13. The wound cleaning adhesive patch as claimed in claim 10, wherein the surfactant is selected from the group consisting of soap and detergent.
